SEQUENCE LISTING (22 sequences)

SEQ ID NO: 1 - protein, 12 aa, Artificial Sequence
  Antiviral peptide
KKKKYRNIRRPG

SEQ ID NO: 2 - protein, 18 aa, Artificial Sequence
  Antiviral peptide
TLKPIFKLPLGINITNFR

SEQ ID NO: 3 - protein, 10 aa, Artificial Sequence
  Antiviral peptide
AKPSYPPTYK

SEQ ID NO: 4 - protein, 6 aa, Artificial Sequence
  Antiviral peptide
SLIGRL

SEQ ID NO: 5 - protein, 16 aa, Artificial Sequence
  Antiviral peptide
WLVFFVIFYFFRRRKK

SEQ ID NO: 6 - protein, 48 aa, Artificial Sequence
  Antiviral peptide
ARGARGLYSLYSTRPLEAVALPHEPHEVALILEPHETYRPHEPHEARG

SEQ ID NO: 7 - protein, 20 aa, Artificial Sequence
  Antiviral peptide
RRKKAAVALLPAVLLALLAP

SEQ ID NO: 8 - protein, 17 aa, Artificial Sequence
  Anti-viral motif
KLLLKLLKKLLKLLKKK

SEQ ID NO: 9 - protein, 10 aa, Artificial Sequence
  adhesive protein
AKPSYPPTYK

SEQ ID NO: 10 - protein, 20 aa, Artificial Sequence
  adhesive protein
AKPSYPPTYKAKPSYPPTYK

SEQ ID NO: 11 - protein, 60 aa, Artificial Sequence
  adhesive protein
AKPSYPPTYKAKPSYPPTYKAKPSYPPTYKAKPSYPPTYKAKPSYPPTYKAKPSYPPTYK

SEQ ID NO: 12 - protein, 39 aa, Artificial Sequence
  adhesive protein
EVHACKPNPCKNNGRCYPDGKTGYKCKCVGGYSGPTCAC

SEQ ID NO: 13 - protein, 52 aa, Artificial Sequence
  adhesive protein
ADYYGPKYGPPRRYGGGNYNRYGGSRRYGGYKGWNNGWKRGRWGRKYYEFEF

SEQ ID NO: 14 - protein, 46 aa, Artificial Sequence
  adhesive protein
ADYYGPKYGPPRRYGGGNYNRYGRRYGGYKGWNNGWKRGRWGRKYY

SEQ ID NO: 15 - protein, 60 aa, Artificial Sequence
  adhesive protein
GHVHRHRVLHKHVHNHRVLHKHLHKHQVLHGHVHRHQVLHKHVHNHRVLHKHLHKHQVLH

SEQ ID NO: 16 - protein, 75 aa, Artificial Sequence
  adhesive protein
SSEEYKGGYYPGNAYHYHSGGSYHGSGYHGGYKGKYYGKAKKYYYKYKNSGKYKYLKKARKYHRKGYKKYYGGSS

SEQ ID NO: 17 - protein, 76 aa, Artificial Sequence
  adhesive protein
SSEEYKGGYYPGNTYHYHSGGSYHGSGYHGGYKGKYYGKAKKYYYKYKNSGKYKYLKKARKYHRKGYKKYYGGGSS

SEQ ID NO: 18 - protein, 71 aa, Artificial Sequence
  adhesive protein
YDDYSDGYYPGSAYNYPSGSHWHGHGYKGKYYGKGKKYYYKFKRTGKYKYLKKARKYHRKGYKKHYGGSSS

SEQ ID NO: 19 - protein, 76 aa, Artificial Sequence; identical to SEQ ID NO: 17
  adhesive protein

SEQ ID NO: 20 - protein, 99 aa, Artificial Sequence
  adhesive protein
GGGNYRGYCSNKGCRSGYIFYDNRGFCKYGSSSYKYDCGNYAGCCLPRNPYGRVKYYCTKKYSCPDDFYYYNNKGYYYYNDKDYFNCGSYNGCCLRSGY

SEQ ID NO: 21 - protein, 194 aa, Artificial Sequence
  adhesive protein
AKPSYPPTYKAKPSYPPTYKAKPSYPPTYKAKPSYPPTYKAKPSYPPTYKAKPSYPPTYKSSEEYKGGYYPGNAYHYHSGGSYHGSGYHGGYKGKYYGKAKKYYYKYKNSGKYKYLKKARKYHRKGYKYYGGSSAKPSYPPTYKAKPSYPPTYKAKPSYPPTYKAKPSYPPTYKAKPSYPPTYKAKPSYPPTYK

SEQ ID NO: 22 - protein, 194 aa, Artificial Sequence; identical to SEQ ID NO: 21
  adhesive protein

(19) United States
(12) Patent Application Publication
(10) Pub. No.: US 2023/0201405 A1
(43) Pub. Date: Jun. 29, 2023

(54) ANTIVIRAL FILTER MEDIUM, AND AIR FILTER UNIT AND AIR CONDITIONER INCLUDING SAME

(71) Applicant: AMOGREENTECH CO., LTD., Gimpo-si, Gyeonggi-do (KR)

(72) Inventors: Seoung Hoon LEE, Gimpo-si, Gyeonggi-do (KR); Seon Ho JANG, Gimpo-si, Gyeonggi-do (KR); In Yong SEO, Gimpo-si, Gyeonggi-do (KR); Chan KIM, Gimpo-si, Gyeonggi-do (KR); Dong Sik SEO, Gimpo-si, Gyeonggi-do (KR)

(73) Assignee: AMOGREENTECH CO., LTD., Gimpo-si, Gyeonggi-do (KR)

(21) Appl. No.: 17/999,446

(22) PCT Filed: May 24, 2021
(86) PCT No.: PCT/KR2021/006418

§ 371 Date: Nov. 21, 2022

(30) Foreign Application Priority Data
May 22, 2020 (KR) 10-2020-0061552
May 22, 2020 (KR) 10-2020-0061553

(51) Int. Cl. A61L 9/16; C07K 14/00; C07K 7/04; C09D 189/00; F24F 8/10; B01D 39/16

(52) U.S. Cl. CPC A61L 9/16; C07K 14/001; C07K 7/04; C09D 189/00; F24F 8/10; B01D 39/1607; C07K 2319/00; B01D 2239/0492; B01D 2239/0442

(57) ABSTRACT

Provided is an antiviral filter medium. An antiviral filter medium according to one embodiment of the present invention includes a first member provided with an antiviral coating layer formed of fibers and including, on part or all of the outer surface of the fibers, an antiviral fusion protein in which an antiviral motif is bound to an adhesive protein. Accordingly, the antiviral filter medium exhibits antiviral properties, is excellent in filtration efficiency and ventilation amount (or flow rate), and has low pressure loss. In addition, the antiviral filter medium is characterized in that the coating layer exhibiting antiviral properties retains adhesiveness for a long period of time after being attached to the surface. Moreover, the antiviral filter medium can retain antiviral activity for a long time without loss of the antiviral activity due to external conditions during production, storage, and use.

### CROSS-REFERENCE TO RELATED APPLICATIONS

This application is a 35 U.S.C. 371 National Phase Entry Application from PCT/KR2021/006418 filed May 24, 2021, which claims priority to and the benefit of Korean Patent Application No. 10-2020-0061552 and 10-2020-0061553, both filed on May 22, 2020, the disclosures of which are incorporated herein by reference in their entirety.

### TECHNICAL FIELD

The present invention relates to a filter medium, and more particularly, to an antiviral filter medium.

### BACKGROUND

Public health is threatened by the proliferation of microbial and viral pathogens. SARS which began to spread in early 2003, MERS which began to infect people in 2012 and also caused 186 cases in South Korea in 2015, and COVID-19 which began in late 2019 and has caused a worldwide epidemic to date, are all caused by viruses, and the spread of such viruses has had a huge impact on individual and community life, from private gatherings to schools, workplaces, and the like, where communal living is required.

Pathogenic microorganisms such as bacteria and viruses contaminate the surfaces of objects and air with pollutants such as droplets discharged from infected people who carry the pathogenic microorganisms, and through the body or clothing that comes into contact with the surfaces of contaminated objects or through inhaling polluted air, microorganisms may be transmitted into the human body.

In particular, the potential for transmission of microorganisms such as viruses through the air shared by many people in enclosed spaces is very high, and even though the air in the closed space is purified with a general filter, the pathogenic microorganisms present in the air are not filtered out or killed, and thus are discharged into the air again, so that there is a problem in that it is difficult to prevent the spread of pathogenic microorganisms through the air with air purifiers or filters that have been commercialized to date. Accordingly, there is a big risk of mass infections due to vehicles such as buses and private cars, transportation means such as subways and trains, central air conditioners installed in various public buildings, and air conditioners such as air purifiers disposed in various rooms.

Further, it is also difficult to control water contaminated with saliva such as droplets discharged from infected people, through commercialized water treatment devices to date, so that there is a big concern about transmission through viruses present in purified water.

Therefore, although there have been recent attempts to provide filters with antipathogenic microbial formulations capable of killing pathogenic microorganisms such as viruses, there is a problem in that the effect of killing pathogenic microorganisms lasts for a very short time as the air or water passing through a filter at a very high pressure causes the antipathogenic microbial formulation to be easily detached from the filter.

Therefore, there is an urgent need for developing a filter medium whose effect can last for a long time while having antiviral properties capable of killing viruses present in air or water.

### SUMMARY OF THE INVENTION

The present invention has been devised in consideration of the above points, and an object of the present invention is to provide an antiviral filter medium that has antiviral properties, is excellent in filtration efficiency and ventilation amount (or flow rate), and has low pressure loss.

In addition, another object of the present invention is to provide an antiviral filter medium that can retain antiviral activity for a long time without loss of the antiviral activity due to external conditions during production, storage, and use while being characterized in that the coating layer exhibiting antiviral properties retains adhesiveness for a long period of time after being attached to the surface, and an antiviral filter unit, air conditioner or water treatment device including the same. To solve the above-described problems, the present invention provides an antiviral filter medium including a first member having fibers provided with an antiviral coating layer formed on part or all of the outer surface of the fibers, wherein the antiviral coating layer includes an antiviral fusion protein in which antiviral motif is bound to an adhesive protein.

According to one embodiment of the present invention, the antiviral motif may target a protein that binds to a host cell receptor to disable or disrupt the protein, or may perform the function of disrupting the viral membrane.

Furthermore, the adhesive protein may be a mussel-derived adhesive protein.

Further, the antiviral motif is any one peptide selected from the group consisting of amino acid sequences of SEQ ID NOS: 1 to 8, or a peptide in which one or more amino acid sequences selected from the above group are linked, and the adhesive protein may be any one protein selected from the group consisting of amino acid sequences of SEQ ID NOS: 9 to 22, or a protein in which one or more amino acid sequences selected from the above group are linked.

In addition, the antiviral coating layer may be formed on a fiber by aggregating particles formed of the antiviral fusion protein.

Furthermore, the antiviral coating layer may be formed through an antiviral coating composition including an antiviral fusion protein and an aggregation-inducing component including a carbodiimide-based coupling agent and a hydroxy succinimide-based reactive agent.

Further, the adhesive protein contains a DOPA residue, and the antiviral fusion protein may be immobilized on a fiber through the DOPA residue. In this case, the DOPA residue may be a DOPA residue into which some or all tyrosine residues of the adhesive protein are modified through an enzyme.

In addition, the fibers forming the first member may have an average diameter of 0.05 to 1 μm, a basis weight of 2.5 g/m² or less, and an average pore diameter of 2.5 μm or less.

Furthermore, a porous second member disposed on one side or both sides of the first member may be further included.

Further, a porous second member disposed on one side of the first member and performing a supporting function and a porous third member which is disposed on the other side of the first member facing the one side and electrostatically treated may be further included.

In addition, a porous fourth member disposed between the first member and the third member and including a silver wire so as to have an antibacterial function, or including a heat-fusible fiber for attaching the first member and the third member may be further included.

Furthermore, the present invention provides an air filter unit including an antiviral filter medium according to the present invention, which is bent so as to alternately form peaks and valleys in one direction, and a filter frame surrounding the antiviral filter medium.

According to one embodiment of the present invention, a filter frame disposed so as to surround at least one surface of the filter medium may be further included.

Further, the present invention provides an air conditioner including the antiviral filter medium according to the present invention.

The antiviral filter medium according to the present invention exhibits antiviral properties, is excellent in filtration efficiency and ventilation amount (or flow rate), and has low pressure loss. In addition, the antiviral filter medium is characterized in that the coating layer exhibiting antiviral properties retains adhesiveness for a long period of time after being attached to the surface. Moreover, the antiviral filter medium can retain antiviral activity for a long time without loss of the antiviral activity due to external conditions during production, storage, and use.

### BRIEF DESCRIPTION OF THE DRAWINGS

FIG. 1 is a cross-sectional schematic view of a coronavirus.

FIG. 2 is a schematic view related to targets by which antiviral motifs can act against coronaviruses.

FIGS. 3 to 6 are schematic views of antiviral filter media according to various embodiments of the present invention.

FIGS. 7 and 8 are perspective views of masks equipped with an antiviral filter medium according to one embodiment of the present invention.

FIGS. 9 to 11 are views of air filter units equipped with an antiviral filter medium according to one embodiment of the present invention.

FIG. 12 is a cross-sectional view of a ventilation-type air purifier equipped with an air filter unit according to one embodiment of the present invention.

FIGS. 13 and 14 are cross-sectional views of antiviral filter media for water treatment according to various embodiments of the present invention.

FIG. 15 is a perspective view of a flat plate-type filter unit equipped with an antiviral filter medium according to one embodiment of the present invention.

FIG. 16 is a perspective view of a portable water purification pouch equipped with an antiviral filter medium according to one embodiment of the present invention.

### DETAILED DESCRIPTION

Hereinafter, embodiments of the present invention will be described in detail with reference to the accompanying drawings such that a person with ordinary skill in the art to which the present invention pertains can easily carry out the present invention. The present invention can be embodied in various forms, and is not limited to the embodiments described herein. In order to clearly describe the present invention in the drawings, parts not related to the description are omitted, and the same reference numerals are added to the same or similar constituent elements throughout the specification.

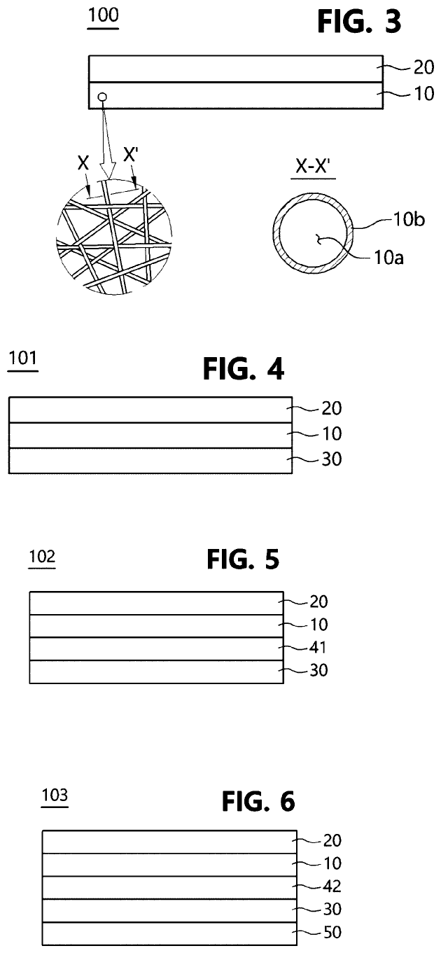

Referring to FIG. 3, an antiviral filter medium 100 according to the present invention includes a first member 10 provided with an antiviral coating layer 10b formed of fibers 10a and including, on part or all of the outer surface of the fibers 10a, an antiviral fusion protein in which an antiviral motif is bound to an adhesive protein, and may further include a porous second member 20 having a supporting function on one surface of the first member 10.

First, the first member 10 having antiviral properties will be described. The first member 10 is formed of fibers provided with an antiviral component and has a porous structure. For example, the first member 10 may be a fiber web formed of fibers 10a in which an antiviral coating layer 10b is formed on some or all of the outer surfaces of the fibers 10a, and more specifically, may have a three-dimensional network structure formed of fibers 10a.

The fiber 10a may be a known fiber-forming component that can be implemented in the shape of a fiber, and may preferably be a component that can be electrospun. The fiber-forming component may include one or more components selected from the group consisting of a fluorine-based compound, polyacrylonitrile (PAN), polyurethane, polyester, polyamide and polyethersulfone (PES). The fluorine-based compound may include, for example, one or more selected from the group consisting of polytetrafluoroethylene (PTFE)-based, tetrafluoroethylene-perfluoroalkyl vinyl ether copolymer (PFA)-based, tetrafluoroethylene-hexafluoropropylene copolymer (FEP)-based, tetrafluoroethylene-hexafluoropropylene-perfluoroalkyl vinyl ether copolymer (EPE)-based, tetrafluoroethylene-ethylene copolymer (ETFE)-based, polychlorotrifluoroethylene (PCTFE)-based, chlorotrifluoroethylene-ethylene copolymer (ECTFE)-based and polyvinylidenefluoride (PVDF)-based compounds. Preferably, the fiber-forming component has a low preparation cost, facilitates mass production of nanofibers through electrospinning, easily implements uniform fineness by electrospinning compared to other materials, and may include polyvinylidene fluoride (PVDF) in terms of excellent mechanical strength and chemical resistance. Furthermore, when PVDF is included, the weight average molecular weight of the PVDF may be 10,000 to 1,000,000, preferably 300,000 to 60,000, but is not limited thereto.

Furthermore, the fiber-forming component may include polyacrylonitrile (PAN), which has excellent wettability with respect to an antiviral coating composition including an antiviral component to be described below, and excellent adhesion between the surface of the fiber-forming component and the antiviral component. In particular, when viruses contained in fine liquid droplets of saliva pass through a filter medium, there is an advantage in that the viruses can be killed with better efficiency when polyacrylonitrile is included as part or all of the fiber-forming component in consideration of the interaction between the droplets and the fiber surface.

Further, the fibers 10a may have an average diameter of 0.05 to 1 μm, as another example, 0.05 to 0.5 μm, and more preferably 0.1 to 0.4 μm. When the average fiber diameter of the fibers 10a is excessively small, the degree of ventilation may be decreased and pressure loss may be increased, and when the average fiber diameter is excessively large, filtration efficiency may be decreased.

In addition, the first member 10 may have a basis weight of 0.4 to 2.5 g/m² or less, and as another example, 0.5 to 2 g/m². When the basis weight is less than 0.4 g/m², filtration efficiency may be decreased, or filtration efficiency may be decreased after long-term use, or uniform filtration efficiency may not be exhibited, and when the basis weight exceeds 2.5 g/m², the degree of ventilation may be decreased and pressure loss may be increased. Furthermore, the average pore diameter may be 0.3 to 2.5 μm, and preferably, the average pore diameter may be 0.5 to 2 μm. When the average pore diameter of the first member 10 is less than 0.3 μm, the degree of ventilation may be decreased and pressure loss may be increased, and when the average pore diameter exceeds 2.5 μm, filtration efficiency may be decreased, or filtration efficiency may be decreased after long-term use.

Further, the first member 10 may have a degree of ventilation of 4 to 35 cfm, preferably 4.5 to 30 cfm. When the degree of ventilation of the first member 10 is less than 4 cfm, pressure loss may be increased, and when the degree of ventilation exceeds 35 cfm, filtration efficiency may be decreased, or filtration efficiency may be decreased after long-term use, or uniform filtration efficiency may not be exhibited.

Meanwhile, the first member 10 may be formed by spinning a spinning solution containing a water repellent/oil repellent. In this case, the first member 10 may be more advantageous when installed in a filtration device that is applied to an outdoor environment, and may be more advantageous in terms of excellent degree of ventilation and filtration efficiency as well as water repellency/oil repellency.

Next, the antiviral coating layer 10b covering part or all of the outer surface of the above-described fiber 10a will be described.

The antiviral coating layer 10b is formed through an antiviral coating composition including an antiviral component including an antiviral fusion protein in which an antiviral motif is bound to an adhesive protein.

The antiviral component includes an antiviral fusion protein, and the antiviral fusion protein is formed by allowing an antiviral motif to bind to an adhesive protein.

The antiviral motif may be a motif that functions to suppress viral proliferation, extinguish a virus itself, or block infection by participating in a mechanism by which a host is infected by the virus.

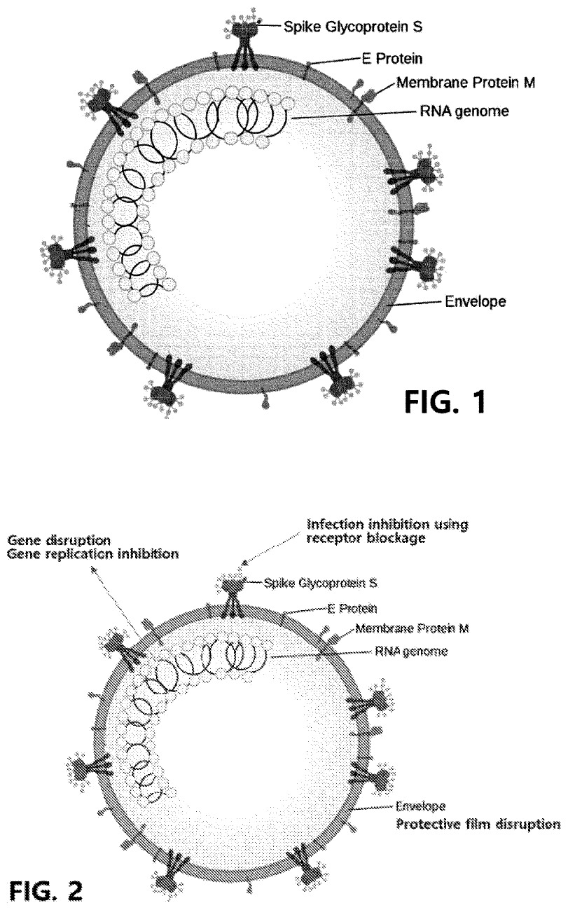

For example, referring to FIGS. 1 and 2, the antiviral motif may have a function of directly or indirectly disrupting the outer membrane, which is the protective film of the virus. Alternatively, when a virus infects a host cell, the antiviral motif may have a function of directly or indirectly disrupting a protein (for example, a spike protein of a coronavirus) bound to the host cell, or directly or indirectly disabling the protein. Here, the direct or indirect means that the antiviral motif directly performs the corresponding function or participates in the initial or intermediate process in ultimately performing the corresponding function.

The antiviral motif can be used without limitation as long as the antiviral motif is a known motif that is known to have an antiviral effect such as destruction or inactivation of the above-described virus. For example, the antiviral motif may be any one peptide selected from the group consisting of amino acid sequences of SEQ ID NOS: 1 to 8, a peptide in which one or more amino acid sequences selected from the above group are linked, or a peptide including one or more amino acid sequences selected from the above group as a basic sequence. For example, the motifs according to SEQ ID NOS: 1 and 2 may be useful for SARS coronaviruses, the motifs according to SEQ ID NOS: 3 to 8 may be useful for influenza A viruses, and in addition, the motif according to SEQ ID NO: 7 may also be useful for HSV.

Further, the antiviral motif may be, for example, a peptide with 3 to 100 amino acids, more preferably 3 to 20 amino acids.

In addition, the virus which the antiviral motif targets is not limited as long as the virus is a known virus, and non-limiting examples thereof include JV, HSV, HIV, IPNV, VHSV, SHRV, HCMV, IAV, Japanese encephalitis virus, Ebola virus, rhinovirus, adenovirus, measles virus, hepatitis B virus, influenza A, and the like.

The above-described antiviral motif itself may be included in the coating composition to treat the surface of a fiber 10a, but it is not easy to immobilize the antiviral motif alone on the surface of the fiber 10a for a long time. Accordingly, the present invention is implemented in the form of a fusion protein in which the antiviral motif is bound to a conjugated protein. The adhesive proteins may function as an adhesive component that provides adhesion between the antiviral motif and the surface of the fiber 10a. Meanwhile, when protein is used as an adhesive component, there is an advantage in that the coating composition can be used for the use of an air filter, a mask, and a filter for a water purifier, and the like which are filter media capable of directly affecting the human body because the protein is non-toxic compared to a polymer-based adhesive component.

Furthermore, the bond between the antiviral motif and the adhesive protein may be a covalent bond, and more specifically, the antiviral motif may be bound to the carboxy terminus, the amino terminus, or both the carboxy terminus and the amino terminus of the adhesive protein by a peptide bond. Meanwhile, the antiviral motif and the adhesive protein may be bound by a known method, and for example, may be prepared by a recombinant protein production method using Escherichia coli. Meanwhile, the adhesive protein and the antiviral motif may be directly bound by a covalent bond, but the bond is not limited thereto, and it should be noted that the adhesive protein and the antiviral motif may be indirectly bound by a covalent bond, and the like by adding a third material as a spacer.

Further, the adhesive protein can be used without limitation as long as the adhesive protein is a protein having a known adhesion function, but may be a mussel-derived adhesive protein, and a known adhesive protein commonly called a mussel-derived adhesive protein can be used without limitation. Preferably, the adhesive protein may include any one protein selected from the group consisting of amino acid sequences of SEQ ID NOS: 9 to 22 or a protein in which one or more amino acid sequences selected from the above group are linked.

In addition, the antiviral component may further include a heterologous material having antiviral functions in addition to the above-described antiviral fusion protein. The heterologous material may be a known organic material or inorganic material. For example, the heterologous material may be an inorganic material with a substituent having proton-donating or proton-accepting properties disposed on the surface with which the virus is brought into contact, and specific examples thereof include phosphate compounds of titanium group elements such as zirconium phosphate, hafnium phosphate, and titanium phosphate and inorganic phosphate compounds such as aluminum phosphate and hydroxyapatite (phosphate minerals); inorganic silicate compounds such as magnesium silicate, silica gel, aluminosilicate, sepiolite (hydrous magnesium silicate), montmorillonite (silicate mineral), and zeolite (aluminosilicate); alumina, titania, hydrous titanium oxide, and the like. Alternatively, the heterologous material may be a metal such as silver or a salt containing ions thereof.

Furthermore, the antiviral coating composition may further include a solvent which dissolves the above-described antiviral fusion protein or a buffer solution which stabilizes the above-described antiviral fusion protein. The solvent may be water and/or an organic solvent, and 20 to 100 mM Tris or a sodium bicarbonate buffer solution with a pH of 8 to 8.5 may be used.

Further, the above-described antiviral fusion protein may be contained at a concentration of 0.001 to 1 mg/ml, and as another example, 0.001 to 0.2 mg/ml in the antiviral coating composition, and when the above-described antiviral fusion protein is contained at a high concentration, antiviral properties may be improved, but there is a concern that the porous structure of the member may be blocked.

Meanwhile, among antiviral fusion proteins, an adhesive protein, particularly, a mussel-derived adhesive protein is known to have adhesion properties itself, but as a result of studies by the present inventors, when these adhesive proteins are used as they are, they may exhibit no or insignificant levels of adhesion (or cohesion) properties, making it difficult to immobilize the antiviral motif on the surface of the fiber 10a. Accordingly, the antiviral fusion protein in the antiviral coating composition, particularly, the adhesive protein among the antiviral fusion proteins may contain a DOPA residue in order to exhibit more improved adhesion properties with the surface of the fiber 10a. Alternatively, the antiviral coating composition may further contain an aggregation-inducing component including a carbodiimide-based coupling agent and a hydroxy succinimide-based reactive agent.

First, an antiviral coating composition in which the adhesive protein contains a DOPA residue in the antiviral fusion protein will be described.

As described above, in the case of an adhesive protein, particularly, a mussel-derived adhesive protein by itself, it is difficult for the adhesive protein itself to exhibit sufficient adhesive and cohesive properties. However, when a DOPA residue is contained, there is an advantage in that the antiviral motif can be easily and strongly immobilized on the surface of the target through the DOPA residue. The DOPA residue may be provided in an antiviral fusion protein through modification, the modification modifies some or all of the tyrosine residues contained within the adhesive protein into DOPA residues, and such modification may be performed appropriately using known methods. For example, the modification may be performed using an enzyme, and the enzyme may be, for example, tyrosinase.

Specifically, the modification may be performed by including (1) preparing a solution in which an antiviral fusion protein is dissolved in a buffer solution containing ascorbic acid, (2) preparing the solution in an oxygen-saturated state, and then modifying a tyrosine residue in an adhesive protein into a DOPA residue by mixing tyrosinase with the solution and (3) performing desalting with acetic acid.

In this case, in Step (1), the buffer solution contains ascorbic acid as an antioxidant at a concentration of 25 to 100 mM, an antiviral fusion protein may be provided at a final concentration of 0.1 to 10 mg/ml in the solution, and the buffer solution may include 20 to 100 mM sodium acetate and 20 to 100 mM sodium borate.

In addition, Step (2) may be performed by saturating the prepared solution with oxygen in the solution while injecting oxygen for 10 minutes to 1 hour, adding a tyrosinase to a final concentration of 10 to 50 μg/ml, mixing and stirring the resulting mixture under oxygen conditions for 30 minutes to 2 hours, and then terminating the reaction by adding acetic acid to a final concentration of 2 to 10%.

Furthermore, Step (3) may be performed by desalting and concentrating the reaction solution in which the reaction is terminated with a 1 to 10% acetic acid solution.

Further, after the modification is performed up to Step (3), the antiviral fusion protein modified to contain a DOPA residue may be prepared in a powder form by lyophilization.

An antiviral fusion protein containing a DOPA residue prepared by the above-described method may be easily immobilized on the desired surface of the fiber 10a without additional adhesive components, and as other adhesive components are not used, there is an advantage in that it is possible to prevent the deterioration or inactivation of activity due to unintended chemical reactions between other components and antiviral motifs and physical blocking.

However, as the improvement of the adhesion of the antiviral fusion protein by modification into the above-described DOPA residue requires additional cost, time, and effort, the coating composition according to one embodiment of the present invention may further include an aggregation-inducing component including a carbodiimide-based coupling agent and a hydroxy succinimide-based reactive agent. The aggregation-inducing component is a material that introduces an antiviral fusion protein to the surface of a target to be treated, and may improve adhesion between the coating layer of the antiviral fusion protein and the surface of the target compared to the case where the surface of the target is treated with the antiviral fusion protein alone using a typical method. Specifically, the aggregation-inducing component aggregates the antiviral fusion protein into particles, and an antiviral coating layer may be implemented in such a manner that these particles are adsorbed on the surface of the fiber 10a to form aggregates. An antiviral coating composition including an aggregation-inducing component may immobilize an antiviral motif on the surface of a target with improved adhesive strength, may also sustain antiviral performance by preventing or minimizing degradation, denaturation, and the like of the antiviral motif at room temperature for a long period of time, and may improve storage stability.

Meanwhile, it is difficult to see that see that the granular form in which the antiviral fusion protein is aggregated by the aggregation-inducing component is due to a specific chemical bond between the fusion proteins, for example, an amino bond between a carboxyl group and an amine group by a carbodiimide-based coupling agent known in the art, which is because a plurality of hydroxy groups included in an adhesive protein, for example, a mussel-derived adhesive protein may also react with a carbodiimide-based coupling agent. Therefore, it is difficult to see that the granular form formed by the antiviral fusion protein having a plurality of reaction sites according to the present invention is due to a specific reaction and the resulting chemical bond, and it may be seen as a unique result occurring according to the combination between antiviral fusion proteins containing an aggregation-inducing component and an adhesive protein.

The carbodiimide-based coupling agent can be used without limitation in the case of a coupling agent that allows antiviral fusion proteins to bind to each other, and may be, for example, 1-[3-(dimethylamino)propyl]-3-ethylcarboimide hydrochloride (EDC) or N,N′-dicyclohexylcarbodiimide (DCC).

Furthermore, the hydroxy succinimide-based reactive agent is provided to increase the efficiency with which antiviral fusion proteins are aggregated with each other by preventing the antiviral fusion protein coupled with carbodiimide from being hydrated, and may be, for example, one of N-hydroxysuccinimide (NHS) and N-hydroxysulfosuccinimide (Sulfo-NHS) or a mixture thereof.

The aggregation-inducing component may include the carbodiimide-based coupling agent and the hydroxy succinimide-based reactive agent at a weight ratio of 1:0.5 to 20, more preferably 1:0.5 to 10, and even more preferably 1:0.5 to 3. When they are not included at an appropriate ratio, it is difficult to achieve the intended effect of the present invention, and there is a concern that when the durability and storage periods of the implemented antiviral coating layer are extended, the activity of the antiviral motif deteriorates.

Further, the aggregation-inducing component may further include sodium acetate, a phosphate buffer solution, or an MES buffer solution as an active component to improve reactivity. In this case, the active component may be included in an amount of 20 to 50 parts by weight with respect to 100 parts by weight of the total weight of the carbodiimide-based coupling agent and the hydroxy succinimide-based reactive agent, and this may be more advantageous in achieving the object of the present invention.

Meanwhile, the above-described aggregation-inducing component may be added to the coating composition as a liquid phase dissolved in a solvent, and in this case, water or an organic solvent may be used as the solvent, and preferably, water may be used, and ethanol may be further included as a solvent in terms of increasing the volatilization rate of the solvent in the coating composition. When the evaporation of the solvent is delayed after the treatment of the coating composition, the antiviral coating composition may flow from the fiber 10, so that there is a concern that it may be difficult to form an antiviral coating layer with desired content and thickness and pores in the porous structure may be clogged.

Meanwhile, when the antiviral coating composition further includes an aggregation-inducing component, the reaction between the antiviral fusion protein and the aggregation-inducing component may continue to occur in an antiviral coating composition state before treatment of the surface of the fiber 10a with the composition due to the aggregation-inducing component, and when the target surface is treated with the antiviral coating composition after the above reaction has excessively progressed beyond the target level, it is difficult to form an antiviral coating layer, or even though the antiviral coating layer is formed, adhesive strength is weak, or the antiviral coating layer is formed to have a rough coating surface, or it is not easy to coat the antiviral coating composition such as a portion which is not coated is present, and the quality of a prepared antiviral coating layer may be poor. In addition, it may be difficult for the antiviral motif in the antiviral coating layer to be exposed to the outside, so that there is a concern that antiviral characteristics deteriorate. In addition, there is a concern that pores in the porous structure may be clogged.

Accordingly, it is preferred that the antiviral coating composition further include a delaying component capable of delaying the reaction between the antiviral fusion protein and the aggregation-inducing component, or the coating composition is stored under conditions capable of delaying the reaction, as an example, under a low-temperature condition which is 0 to 15° C., as another example, 0 to 10° C.

Alternatively, as another exemplary embodiment, the antiviral coating composition is prepared using a first solution including an antiviral component and a second solution including an aggregation-inducing component, and then the first solution and the second solution are mixed according to the timing of treatment of the surface of the fiber 10a, and then the surface of the target may be immediately treated with the mixture, or the surface of the fiber 10a may be treated with the mixture after imparting a predetermined reaction time after mixing of the components.

When the process of preparing the coating composition containing the above-described aggregation-inducing component is looked at in detail, a second solution in which the aggregation-inducing component including the carbodiimide-based coupling agent and the reactive agent is dissolved in a solvent and a first solution in which the antiviral fusion protein is dissolved are each prepared, and then these solutions may be mixed at a predetermined content.

The first solution may be prepared by dissolving the prepared antiviral fusion protein in a solvent, for example, water.

In addition, the second solution may be prepared by mixing a carbodiimide-based coupling agent, a hydroxy succinimide-based reactive agent and a solvent, for example, water and/or ethanol, or prepared by preparing each of a mixed solution of a carbodiimide-based coupling agent and a solvent and a mixed solution of a hydroxy succinimide-based reactive agent and a solvent, and then mixing these mixed solutions.

In this case, the above-described active component may be included in the second solution, and for example, the second solution may be an activated solution obtained by mixing a carbodiimide-based coupling agent, a hydroxy succinimide-based reactive agent, and the active component, and then reacting the mixture for 1 to 60 minutes. Alternatively, the second solution may also be prepared by preparing each of a first mixed solution of a carbodiimide-based coupling agent and an active component and a second mixed solution of a hydroxy succinimide-based reactive agent and an active component, and then mixing these mixed solutions. In this case, two mixed solutions may be mixed with the first solution immediately after being mixed, but as another example, the two mixed solutions are mixed, and then the second solution may be prepared by inducing a reaction for 30 to 60 minutes. Next, a step of mixing the prepared first and second solutions may be performed. In this case, the mixing ratio of the first solution and the second solution may be appropriately changed in consideration of a specific method of treating the surface with the antiviral coating composition, the thickness of a coating layer to be formed, the degree of antiviral activity, and the like. As an example, the first solution and the second solution may be mixed by adjusting the content, such that the total weight of the carbodiimide-based coupling agent and the hydroxy succinimide-based reactive agent is 50 to 200 parts by weight, as another example, 80 to 120 parts by weight, with respect to 100 parts by weight of the antiviral fusion protein. When the total weight of the carbodiimide-based coupling agent and the hydroxy succinimide-based reactive agent is less than 50 parts by weight, it may be difficult to implement a granular form, so that the coatability on the surface of the fiber 10a may deteriorate. Furthermore, when the total content exceeds 200 parts by weight, the coating layer may be peeled off.

Meanwhile, after the mixing of the first solution and the second solution prepared as described above is performed, an aging step of inducing a reaction between the antiviral fusion protein in the first solution and the aggregation-inducing component in the second solution may be further performed.

Here, inducing a predetermined reaction means that the antiviral fusion protein is initiated to aggregate on the antiviral coating composition, or the surface of the target is treated in a state in which the antiviral fusion protein has already been aggregated to form particles having a predetermined size, and is not limited thereto, and it should be noted that an aggregation reaction may be induced by treating the surface of the fiber 10a with the antiviral coating composition immediately after mixing the solutions. The aging step may be controlled by the content of the fusion protein and the aggregation-inducing component, the method of treating the target with the antiviral coating composition, and the like, and may be performed, as an example, for more than 0 to 300 minutes, and as another example, for 30 to 60 minutes. The aging time may also vary depending on the temperature conditions during aging, and the aging time during aging at low temperature may be increased. For example, when the surface of the fiber 10a is coated with the antiviral coating composition using an impregnation method at room temperature, for example, 20 to 25° C., the aging time may be, as an example, 10 minutes or more, and as another example, 30 minutes, or 40 minutes or more. As another example, when the antiviral coating composition is electrosprayed, the aging time may be as an example, within 10 minutes, and as another example, within 8 minutes, 6 minutes, 4 minutes, and 2 minutes, for example, at 20 to 25° C.

In addition to the components described above, the above-described antiviral coating composition may further contain a component such as a dispersant, a leveling agent, a viscosity modifier, and an antifoaming agent, which are contained in a typical coating composition, and the specific types and contents thereof are not particularly limited in the present invention, because the known types may be used by adjusting the content to an appropriate content depending on the purpose.

Further, the antiviral fusion protein in the above-described antiviral coating composition may be immobilized on the surface of a fiber 10a in the first member through the treating of the surface of the fiber 10a with the antiviral coating composition and the drying of the antiviral coating composition.

The surface of the fiber 10a may be treated with the antiviral coating composition through a known coating method, and the known coating method may be, for example, impregnation, spin coating, comma coating, spraying, electrospraying, and the like. In addition, the antiviral coating composition may be implemented to have an appropriate viscosity according to a specific coating method.

When looking specifically at the case where the antiviral coating composition, particularly, the antiviral coating composition further including an aggregation-inducing component is applied onto the surface of the target by electrospraying, the electrospraying may be performed using a known electrospraying device. In this case, the conditions of the electrospraying are as follows: the distance between a tip and a collector may be 10 to 50 cm, the voltage applied to the tip may be 30 to 70 kV, the temperature during spraying may be 20 to 40° C., the relative humidity may be 30 to 50%, and through this, it may be suitable for implementing an antiviral coating layer which is uniform and exhibits the desired effect of the present invention.

Furthermore, only the surface of the fiber 10a present on the surface portion of the first member 10 may be treated with the antiviral coating composition, or the surface of the fiber 10a may be treated with the antiviral coating composition, such that the antiviral coating composition may be brought into contact with the surface of the fiber 10a, regardless of the position of the fibers forming the first member 10. Further, the antiviral coating layer may be formed by appropriately adjusting the concentration of each component in the antiviral coating layer, the aging time, and the like such that the pores retained before treatment are not blocked even after the antiviral coating layer is formed.

In addition, after the surface of the target is treated with the antiviral coating composition, a reaction may be induced for a predetermined time such that the antiviral coating layer is formed. In this case, the reaction time may vary depending on the concentration of the antiviral fusion protein in the coating composition, the concentration of the aggregation-inducing component, the thickness of the desired coating layer, the time taken for the antiviral coating composition to react after penetrating into the first member 10, the temperature, and the like.

The reaction may be completed before the drying step after the treatment of the antiviral coating composition, but is not limited thereto, and it should be noted that the reaction may occur after the treatment of the antiviral coating composition, or the reaction may be completed through a drying step, particularly, a drying step performed by applying heat to be described later in a state in which the reaction is only partially completed.

Thereafter, the antiviral coating composition is dried naturally at room temperature for 1 to 24 hours or dried with hot air and/or an IR lamp at a temperature of 30 to 100° C., so that the antiviral fusion protein may form particles on the surface of the target to implement an antiviral coating layer bound to the surface of the target.

Next, the above-described first member 10 may further include a porous second member 20 that performs a supporting function on one surface thereof.

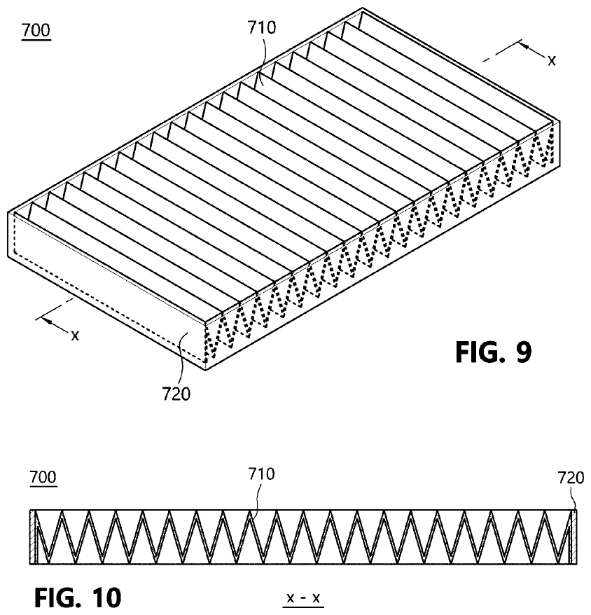

The second member 20 is not particularly limited in the case of a porous member that usually serves as a support, but may preferably be a woven, knitted or non-woven fabric. For example, in the case of the non-woven fabric, it is possible to use a dry non-woven fabric such as a chemically-bonded non-woven fabric, a thermally-bonded non-woven fabric and an air-laid non-woven fabric, or a known non-woven fabric such as a wet non-woven fabric, a spunless non-woven fabric, a needle-punched non-woven fabric or a melt-blown non-woven fabric, and for example, the second member 20 may be a thermally-bonded non-woven fabric. Alternatively, the second member 20 may function to prevent damage to the first member 10 due to bending when the filter medium is bent as illustrated in FIGS. 9 and 10. In such a case, the second member 20 may be, for example, a spunbond non-woven fabric made of a polyester-based material.

Furthermore, since the pore diameter, porosity, basis weight, and the like of the second member 20 may vary in consideration of the desired strength, filtration efficiency, and the like, the present invention is not particularly limited thereto. However, in order to more easily achieve the object of the present invention, it is possible to use a non-woven fabric including fibers with a diameter of 30 to 50 μm, a basis weight of 30 to 150 g/m², and an average pore diameter of 30 to 100 μm.

Further, a material for the second member 20 is not limited. Non-limiting examples thereof may include preferably a synthetic fiber selected from the group consisting of polyester, polypropylene, nylon and polyethylene. However, the second member 20 may include a heat-fusible fiber so as to be able to be attached to the first member 10 without a separate adhesive. The heat-fusible fiber may be low-melting polyester or low-melting polyolefin, may include preferably a low-melting-point polyolefin-based component, and more specifically, may be a core-sheath type composite fiber formed of a sheath including polyethylene and a core including polyethylene with a melting point higher than that of the sheath. The first member 10 may include small fibers 10a with a diameter of 1 μm or less, but when the second member 20 is formed of polyolefin-based fibers, better bonding properties are exhibited when bonded to the first member 10 through thermal fusion compared to the case where a second member formed of polyester-based fibers is used, and it is possible to better respond to an external force applied while air is passing because the second member 20 formed of polyolefin-based fibers is more flexible than a second member formed of highly brittle polyester-based fibers and thus there is an advantage of preventing separation at the bonding interface.

In addition, referring to FIGS. 4 to 6, an antiviral filter medium 101 may further include a third member 30. The third member 30 may be disposed so as to be brought into direct contact with the first member 10 as illustrated in FIG. 4, or may be disposed by interposing a fourth member 41 between the third member 30 and the first member 10 as illustrated in FIG. 5.

Specifically, the third member 30 may be disposed on one side of the first member 10 as illustrated in FIG. 4 to perform a function of protecting the first member 10. Furthermore, the third member 30 may be a member through which external air to be filtered primarily passes, thereby performing a function of improving filtering performance with respect to fine particles in the air. For this purpose, the third member 30 may be a porous member that filters fine dust particles, dust particles and the like included in the air using electrostatic force. The third member 30 may be, for example, a known non-woven fabric, preferably an electrostatically treated porous member, and specifically an electrostatically treated melt-blown non-woven fabric. It should be noted that although the entire porous member can be subjected to electrostatic treatment, only a part of the porous member may be subjected to electrostatic treatment, and a detailed description thereof will be omitted because a known method used when a typical electrostatically treated filter is manufactured may be appropriately employed.

Further, the diameter and basis weight of the fibers contained in the third member 30 may be appropriately adjusted according to the purpose, and in order to secure more improved filtration performance, durability, and the like, the third member 30 includes fibers with an average diameter of 0.5 to 12 μm, preferably 1 to 10 μm, and the basis weight may be 15 to 50 g/m², and as another example, the basis weight may be 20 to 38 g/m². In addition, the average pore diameter may be 20 μm or less, and as another example, it may be 5 to 15 μm, more preferably 7 to 12 μm. When the basis weight of the third member 30 is less than 15 g/m², filtration efficiency may be decreased as a deviation is increased, or uniform filtration efficiency may not be exhibited, and when the basis weight exceeds 40 g/m², the degree of ventilation may be decreased, and pressure loss may be increased. Furthermore, when the average pore diameter of the third member 30 is less than 5 μm, the degree of ventilation may be decreased and pressure loss may be increased, and when the average diameter exceeds 15 μm, filtration efficiency may deteriorate. Further, when the average diameter of the fibers forming the third member 30 is excessively small, the degree of ventilation may be decreased and pressure loss may be increased, and when the average diameter of the fibers is excessively large, filtration efficiency may be decreased.

In addition, the third member 30 may have a degree of ventilation of 25 to 40 cfm, preferably 30 to 36 cfm. When the degree of ventilation is less than 25 cfm, pressure loss may be increased, and when the degree of ventilation exceeds 40 cfm, filtration efficiency may be decreased or uniform filtration efficiency may not be achieved.

Furthermore, the fibers forming the third member 30 may include a synthetic polymer component selected from the group consisting of polyester-based, polyurethane-based, polyolefin-based and polyamide-based polymers, or a natural polymer component including a cellulose-based polymer, and for example, may include polypropylene.

Meanwhile, in the case of antiviral filter media 101, 102, and 103 in which the third member 30 and the first member 10 are combined, a synergistic effect is exhibited in terms of the durability of filtration performance together with the first member 10. Specifically, the first member 10 may form a three-dimensional network structure by accumulating fibers of 1 μm or less. In this case, the first member 10 may be designed to have a pore diameter capable of physically filtering even fine dust particles of PM 2.5 or less, and a flow path may be formed so as to prevent a decrease in the flow rate of passing air. In this case, the first member 10 compensates for a problem of a reduction in collection efficiency due to de-electrification that may occur in the electrostatically treated third member 30, and may function to maintain the initially designed filtration efficiency for a long time.

Specifically, the electrostatically treated third member 30 adsorbs dust particles to the fiber surface using electrostatic force, and as time passes, the electrostatic force decreases, and after 5 months of use, there is a problem in that filtration efficiency may be decreased to 50% or less of the initially designed efficiency, and accordingly, the replacement cycle is very short. However, when the first member 10 is used together with the electrostatically treated third member 30, there is a small decrease in collection efficiency unlike the continuous decrease in collection efficiency when the third member 30 is used alone, and there is an advantage in that collection efficiency can be maintained at 95% or more of the initially designed value even though the decrease in collection efficiency lasts for 5 months or longer.

Next, the fourth member 41 or 42 which may be provided between the first member 10 and the third member 30 will be described.

The fourth member 41 or 42 may be a porous member provided with a silver wire so as to have an antibacterial function while performing the supporting function of a filter medium or provided with heat-fusible fibers for attaching the first member 10 and the third member 30.

First, the case where the fourth member 41 or 42 exhibits an antibacterial function will be described.

The fourth member 41 or 42 can be used without limitation as long as it is a porous member containing a known component in order to exhibit an antibacterial function, and preferably, the fourth member 41 or 42 may include fibers containing silver that exhibits antibacterial characteristics.

The silver-containing fiber is a silver wire consisting of silver alone, or may be a metal wire containing other metals such as copper other than silver, or a plied yarn formed by combining a silver wire and/or a silver-containing metal wire with a typical non-metallic fiber. When the metal wire containing other metals such as copper is described, the metal wire may be linearly formed by mixing metals other than silver with silver in a non-solid solution state, that is, with silver in a non-alloyed state. When metals other than silver are mixed with silver in a non-solid solution state, silver and other metals may be disposed such that silver and other metals within a single-stranded linear region regularly or irregularly occupy a predetermined region, respectively, and for example, it may be a double structure in which silver surrounds the outside of a copper wire to form a layer. In this case, the copper wire may impart excellent flexibility to the silver wire, and the surrounding silver may have an average thickness of 3 to 3200 nm, preferably 5 to 3000 nm. When the average thickness of the surrounding silver is less than 3 nm, the antibacterial function may deteriorate because copper, which is the central metal, is likely to prepared so as to be exposed to the outside, the antibacterial function may further deteriorate because silver is desorbed from the silver wire, or there is a concern that desorbed silver may be inhaled into the respiratory tract of a person using the desorbed silver or may remain on the skin. Further, when the average thickness of the surrounding silver exceeds 3200 nm, the flexibility of the silver wire may deteriorate. Such a double-structured silver wire may be formed by drawing a copper material to a predetermined diameter, integrating the copper material drawn by a cladding process and a silver plate to obtain a double-structured wire in which the silver plate surrounds the outside of the copper material, and obtaining a silver wire by subjecting the double-structured wire to wire drawing processing. Alternatively, the silver wire may be obtained by treating the drawn copper material with a solution containing a liquid-type Ag powder solution to coat the surface of the copper material with Ag having a uniform thickness, and then performing a wire drawing processing. Alternatively, a silver wire may also be obtained by plating the drawn copper material with silver and subjecting the silver-plated drawn copper material to wire drawing processing.

Next, when the form of yarn formed by combining the silver wire with typical non-metallic fibers is described, the silver wire and typical fibers may be a plied yarn implemented by appropriately employing a known manufacturing method in the field of fibers in which two types of fibers are combined and a known arrangement structure of two fibers. In this case, the silver wire used may be a wire consisting only of silver or a metal wire containing silver and other metals. As an example, the plied yarn may be a yarn having a triple-structured cross section, which includes a core yarn, a first covering yarn including a silver wire surrounding the core yarn, and a second covering yarn surrounding the first covering yarn surrounding the core yarn.

The core yarn and the second covering yarn can be used without limitation as long as the core yarn and the second covering yarn are fibers that can be used to improve the flexibility and stretchability of the plied yarn, and preferably, any one selected from a natural fiber and a synthetic fiber may be used, and more preferably, a polyester-based fiber may be used. In addition, the core yarn and the second covering yarn may be formed of mono-filament yarn or a plurality of filament yarns, and may preferably be fibers formed of a plurality of filament yarns. Furthermore, the core yarn and the second covering yarn can be used without limitation as long as the core yarn and the second covering yarn are fibers with a fineness which can be typically used in the art, and preferably, they each may independently have a fineness of 20 to 100 denier (De′), more preferably 30 to 75 De′. There is a concern that when the finenesses of the core yarn and the second covering yarn are each independently less than 20 De′, antibacterial performance and durability may deteriorate due to the single yarn of the silver wire, and when the fineness exceeds 100 De′, stretchability deteriorates, and thus the bonding strength with the first member and the like is decreased, thereby causing interfacial separation.

Further, the second covering yarn may be twisted at a twist number of 350 to 1100 TPM, preferably at 450 to 1000 TPM to be included in the plied yarn. When the twist number of the second covering yarn is less than 350 TPM, durability and antibacterial performance due to the single yarn of the silver wire may deteriorate. In addition, when the twist number exceeds 1100 TPM, stretchability and flexibility may deteriorate, and as the area of the silver wire exposed on the surface may be decreased, antibacterial performance may relatively deteriorate.

Meanwhile, the fourth member 41 or 42 is a member implemented so as to have a porous structure including the above-described silver wire consisting of silver, a metal wire including silver, and/or a plied yarn, and specific examples thereof may be a woven fabric, knitted fabric, non-woven fabric or mesh sheet. In this case, the woven fabric, knitted fabric, non-woven fabric or mesh sheet may further include a natural fiber and/or synthetic fiber, which do/does not contain a silver wire.

Next, a case where the fourth member 41 or 42 has a characteristic of attaching the first member 10 and the third member 30 will be described. In this case, the fourth member 41 or 42 may be a porous member including heat-fusible fibers, and the shape of the porous member may be a woven fabric, knitted fabric or non-woven fabric. The heat-fusible fiber may be typically a fiber commonly referred to as low-melting-point fiber, and part or all of the fiber may include a low-melting-point component, for example, a component having a melting point of 60 to 200° C., and may preferably be a composite fiber including a low-melting-point component. The composite fiber may be arranged such that at least part of the low-melting-point component is exposed to the outer surface by including the support component and the low-melting-point component. For example, the composite fiber may be a sheath-core type composite fiber in which the supporting component forms a core part and the low-melting point component forms a sheath part surrounding the core part, or a side-by-side composite fiber in which the low-melting point component is disposed on one side of the supporting component. The low-melting-point component and the supporting component may preferably be a polyolefin in terms of flexibility and flexibility of the support as described above, and for example, the supporting component may be polypropylene and the low-melting-point component may be polyethylene. However, they are not limited thereto.

Furthermore, for example, the fourth member 41 or 42 may have a basis weight of 25 to 75 g/m², preferably 30 to 70 g/m², and the fibers forming the fourth member 41 or 42 may have an average diameter of 10 to 30 μm, preferably 15 to 25 μm, and an average pore diameter of 30 to 200 μm.

Further, as illustrated in FIG. 6, the antiviral filter medium 103 may further include a fifth member 50, and the fifth member 50 may be disposed on one side of the third member 30 to form the outermost side through which outside air or raw water passes. In particular, the fifth member 50 may have a function of supporting the filter medium 103 and improving water repellency/oil repellency in the use of an air filter through which outside air passes. In this case, the fifth member 43 may preferably be a non-woven fabric, more preferably a dry non-woven fabric such as a chemically-bonded non-woven fabric, a thermally-bonded non-woven fabrics and an air-laid non-woven fabric, or a wet non-woven fabric, a spunless non-woven fabric, a needle-punched non-woven fabric, a spunbond non-woven fabric, or a melt-blown non-woven fabric, and more preferably a spunbond non-woven fabric.

In addition, in order to impart water/oil repellency, the fifth member 50 may be a water repellent/oil repellent spunbond non-woven fabric implemented by including a water/oil repellent together with the above-described polymer in a spinning solution and spinning or implemented through predetermined water repellent/oil repellent treatment.

Furthermore, when the average diameter of the fibers forming the fifth member 50 is excessively small, the degree of ventilation may be decreased and pressure loss may be increased, and when the average diameter of the fibers is excessively large, water repellency/oil repellency may deteriorate or filtration efficiency may be decreased. Further, when the basis weight is excessively low, filtration efficiency may be decreased or uniform filtration efficiency may not be exhibited as a deviation becomes large, and water repellency/oil repellency may deteriorate, and when the basis weight is excessively high, the degree of ventilation may be decreased and pressure loss may be increased.

Meanwhile, in the case of the second member 20 to the fifth member 20 described above, an antiviral coating layer may be further provided as on the first member 10, and through this, it should be noted that an effect of killing virus which is brought into contact with the filter medium may be further maximized.

The above-described antiviral filter medium 100, 101, 102, or 103 may have a filtration efficiency of 99.5% or more, preferably 99.6% or more, more preferably 99.7% or more, and even more preferably 99.8% or more. In addition, the above-described filter medium 100, 101, 102, or 103 may have a pressure loss of 20 to 120 mmH₂O, preferably 25 to 110 mmH₂O. When the filtration efficiency of the filter medium is less than 99.5%, the filter medium cannot exhibit the desired level of filtration efficiency, so that the filter member cannot be applied to masks, air filters, or ventilation-type air purifiers to be described below. Furthermore, when the pressure loss of the filter medium is less than 20 mmH₂O, the filtration efficiency may not be good, and when the pressure loss exceeds 120 mmH₂O, the degree of ventilation may be decreased.

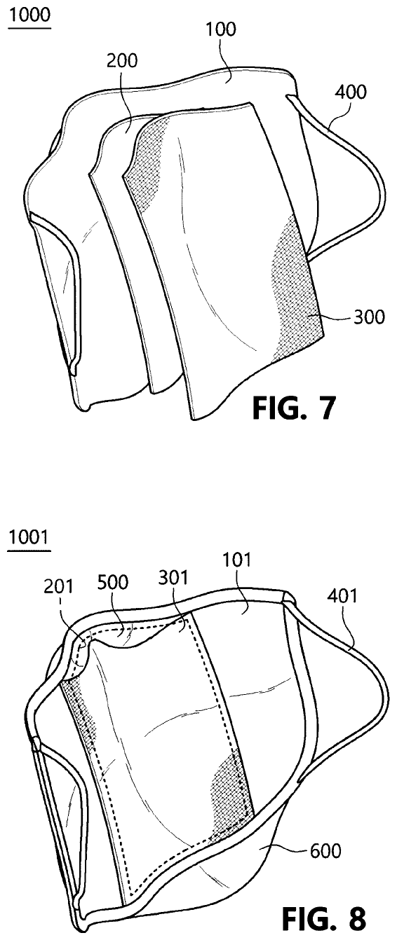

When the above-described antiviral filter medium 100, 101, 102, or 103 is used for air filtration, it may be employed in a mask 1000 or 1001 as specifically illustrated in FIGS. 7 and 8, and in a mask 1000 or 1001 including a first fabric 200 that forms an externally exposed surface, a second fabric 300 that is fixed to at least a part of the first fabric 200 to form an accommodating portion and that adheres to the face of a wearer, and an ear band 400 provided on both sides of the first fabric 200, the antiviral filter medium may be provided in the accommodating portion.

The first fabric 200 may exhibit hydrophobicity, a quick-drying property, water repellency, and the like in order to prevent the generation and growth of bacteria caused by external water, humidity, or a user's saliva. Further, the first fabric 200 can be used without limitation as long as it is formed of a mask outer skin material commonly used in the art, but preferably, may be formed by including any one or more selected from a natural fiber and a synthetic fiber, and more preferably, it is more advantageous to use polyester in terms of exhibiting hydrophobicity, a quick-drying property and water repellency. In addition, the first fabric may be a woven fabric or knitted fabric.

Furthermore, as described above, the second fabric 300 is fixed to at least a part of the first fabric 200 to form an accommodating portion for accommodating the above-described filter medium, and adheres to the face of a wearer. In this case, preferably, the second fabric 300 may be fixed to the first fabric 200 at the upper end and lower end to form an accommodating portion. Meanwhile, the second fabric 300 may have the same or different material and characteristics as the above-described first fabric 200, and may have preferably the same material and characteristics, so that a description thereof will be omitted.

Further, as illustrated in FIG. 8, the mask 1001 may further include a nose pad 500 and a chin pad 600 provided at the lower end of a first fabric 201. The nose pad 500 serves to prevent the inflow of outside air and the discharge of inside air into other regions other than the first fabric 201 and a second fabric 301 when the user breathes, and as the nose pad 500 may have the same material and shape as a known nose pad, the material and shape are not particularly limited in the present invention. In addition, as the chin pad 600 may have the same material and shape as a known nose pad, the material and shape are not particularly limited in the present invention.

Furthermore, as illustrated in FIGS. 9 to 11, an antiviral filter medium 710 or 810 according to the present invention is bent to implement an air filter unit 700 or 800, and through this, the filtration area may be maximized to further improve filtration efficiency.

Further, the air filter unit 700 or 800 according to the present invention may further include a filter frame 720 or 820 disposed so as to surround at least one side of the bent filter member 710 or 810.

The filter frame 720 or 820 is disposed so as to surround at least one side of the bent filter medium 710 or 810 as described above, and preferably, may be disposed so as to surround all sides of the bent filter medium 710 or 820. However, as a known filter frame may be used as the filter frame, the filter frame is not particularly limited in the present invention.

Meanwhile, the filter medium 710 or 810 may be provided while being bent so as to have a peak height of 5 to 55 mm, preferably 10 to 50 mm. When the peak height is less than 5 mm, filtration efficiency may be decreased and pressure loss may be increased as the area of the filter medium provided in the composite filter has a small area, and when the peak height exceeds 55 mm, the peaks may stick to each other to reduce the filtration area, and when the pressure is high, the peaks may be broken or deformed. Meanwhile, in this case, the peak height represents the height from the valley to the peak.

In addition, the air filter unit 700 or 800 according to the present invention may include 70 to 95, preferably 73 to 91, and more preferably 77 to 86 peaks per 300 mm of length, and the filter medium may include 1.3 to 8.5 m, preferably 1.5 to 8.2 m, and more preferably 2 to 7.5 m per 300 mm of length. Since the number of peaks included in the air filter unit 700 or 800 and the length range of the filter medium are satisfied, the specific surface area is high, so that it is possible to simultaneously achieve effects in which removal efficiency is excellent, pressure loss is low, and a decrease in removal efficiency can be prevented.

Furthermore, although a wrinkled structure including a plurality of peaks and valleys has been described as an example, the air filter unit may be implemented in a shape such as a flat plate type, a pleated structure, and a cylindrical type without limitation in shape.

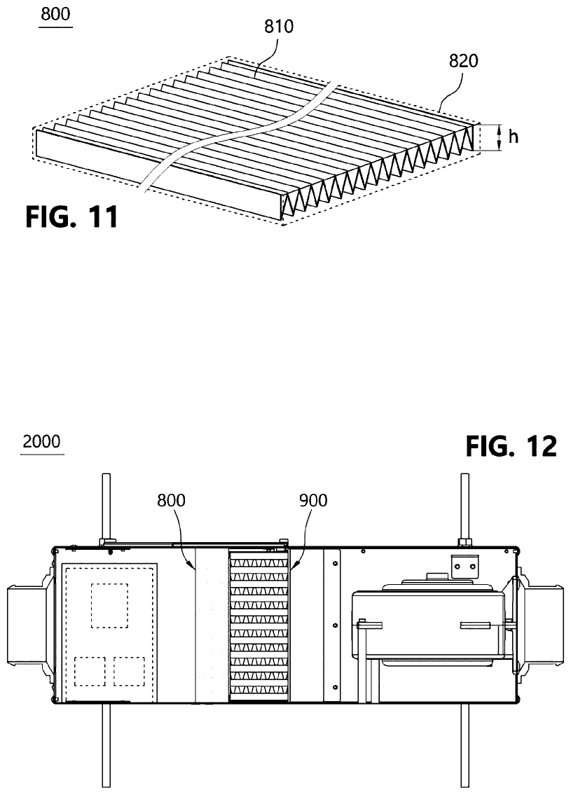

Further, as illustrated in FIG. 12, the air filter unit 800 according to the present invention may be used instead of air filters provided in various known air conditioners. The air conditioner may be an air conditioner provided in a vehicle such as a bus and a private car, a transportation means such as a subway or a train, or an air purifier disposed in various public buildings, or an air purifier installed in various rooms, and as the air conditioner includes a filter medium or air filter unit provided with an antiviral function, it is possible to filter or kill pathogenic microorganisms such as various bacteria or viruses included in the air that flows in from the outside or is circulated indoors.

When a ventilation-type air purifier 2000, which is an example of the air conditioner, is described, the ventilation-type air purifier 2000 may be any one of a wall-mounted type, a stand type, and a ceiling type.

As an example, the ceiling-type ventilation-type air purifier 2000 illustrated in FIG. 12 will be described. The ceiling-type ventilation-type air purifier may be implemented by including a housing, a first blower, a second blower, a heat exchanger 900 and an air filter unit 800. The housing may form an overall outer shape, and may be formed so as to have an internal space in which the first blower, the second blower, the heat exchanger and the filter member may be disposed.

Meanwhile, the housing may include a plurality of ports communicating with the internal space such that indoor air and outdoor air can be discharged after flowing in the inner space. For example, the plurality of ports may include an indoor air inlet for introducing indoor air into the internal space, an indoor air outlet for discharging the indoor air introduced into the internal space to the outside, an outdoor air inlet for inflowing outdoor air into the internal space and an outdoor air outlet for discharging the outdoor air that has flowed in the internal space indoors.

Meanwhile, in the ceiling-type ventilation-type air purifier 2000, the indoor air and the outdoor air respectively introduced into the internal space through the indoor air inlet and the outdoor air inlet are heat exchanged without being mixed with each other, and then may be discharged to the outside from the internal space.

For this purpose, the heat exchanger 900 may be disposed in the internal space such that both the outdoor air sucked through the first blower and the indoor air sucked through the second blower can pass through. In this case, the above-described air filter unit 800 may be disposed on the side of the heat exchanger such that the outdoor air that flows in the internal space is filtered and then flows in the side of the heat exchanger 900.

Accordingly, the outdoor air supplied from the outdoors to the indoors through the operation of the first blower may enhance the quality of the indoor air present indoors by removing pathogenic microorganisms such as viruses from impurities such as yellow sand or fine dust particles in the process of passing the outdoor air through the air filter unit 800.

For a detailed description of the ventilation-type air purifier, it should be noted that the inventors of the application for the ventilation-type air purifier of the present applicant and Korean Patent Application Nos. 10-2020-0070790, 10-2020-0068449 and 10-2020-0070792 may be incorporated by reference.

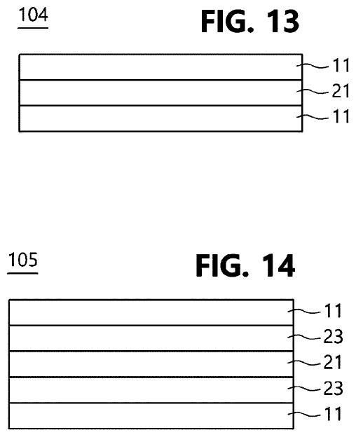

Meanwhile, the above-described antiviral filter medium 100 may be used for water treatment. For example, as illustrated in FIGS. 13 and 14, when used for water treatment, an antiviral filter medium 104 or 105 may have a structure in which a first member 11 is disposed on both surfaces of a second member 21 which performs a supporting function, a sixth member 23 which performs a supporting function is disposed on both surfaces of the second member 21, or the first member 11 is disposed on both surfaces of these laminates, and by employing such a structure, excellent filtration efficiency and an excellent flow rate may be achieved and simultaneously, the structure may be suitable for backwashing performed at high pressure. Meanwhile, the sixth member 23 may have a smaller thickness and basis weight than the second member 21.

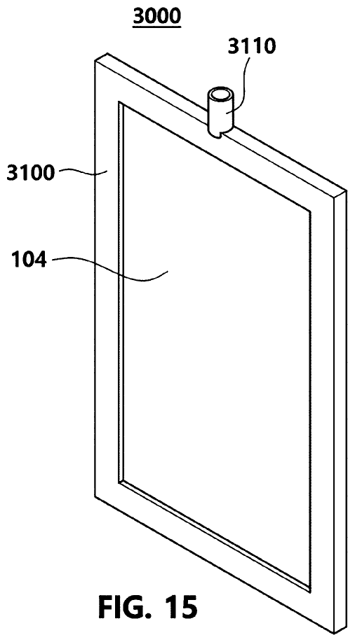

The above-described antiviral filter medium 104 or 105 for water treatment may be employed in a flat plate-type filter unit 3000 as illustrated in FIG. 15. The flat plate-type filter unit 3000 is formed with a flow path through which a liquid to be filtered flows toward the filter medium 104, or the filtrate filtered by the filter medium 104 flows out to the outside, and includes a support frame 3100 which supports the frame of the filter medium 104, and a region of the support frame 3100 may be provided with an inlet 3110 capable of gradating a difference in pressure between the outside and inside of the filter medium 104. In this case, when the flat plate-type filter unit 3000 applies a high-pressure suction force through the inlet 3110, the liquid to be filtered placed outside the filter medium 104 is directed toward the inside of the filter medium 104, and specifically, the filtrate filtered through the first members 11 disposed on both sides flows toward the second member 21, which is a central part, may flow along the flow path formed through the second member 21, and flow in the flow path provided in the outer frame 3100, and then flow out to the outside through the inlet 3110.

Meanwhile, in relation to such filter medium, flat plate-type filter unit and filter device for water treatment, Application Nos. 10-2017-0067945, 10-2017-0068484, 10-2017-0170430 by the present applicant may be incorporated by reference herein.

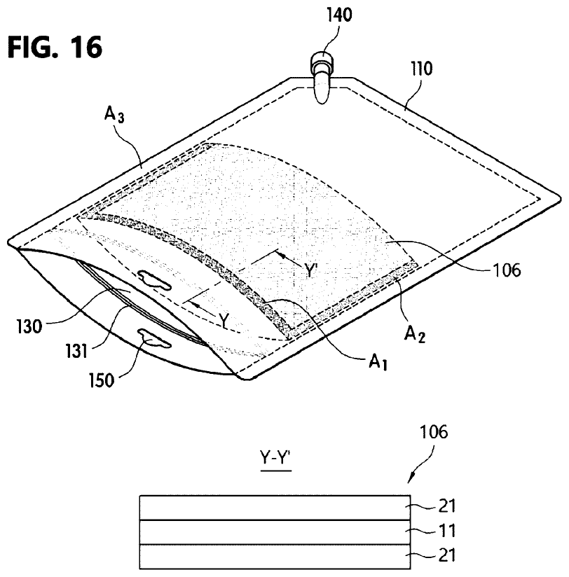

In addition, referring to FIG. 16, the antiviral filter medium 106 may be employed in a portable water purification pouch of a self-weight filtration type. The portable water purification pouch includes a pouch-type body 110 and an antiviral filter medium 106 inside the body 110. The pouch-type body 110 includes an inlet 130 whose one side is completely open to allow water to be treated to flow in and an outlet 140 from which drinking water obtained by filtering the water to be treated is discharged. Furthermore, the filter medium 106 has a pouch shape in which all sides except one side are joined so as to contain the water to be treated that has flowed in from the inlet 130, and the filter medium 120 may be disposed inside the body 110 such that one side of the open filter medium 106 is in fluid communication with the inlet 130 of the body. The antiviral filter medium 106 provided in such a portable water purification pouch implements a pouch shape in which three edges are bonded, and may have a structure in which the second member 21 is provided on both sides of the first member 11 such that one edge portion A₁ or A₂ is easily attached to the pouch-shaped body 110, and in this case, the second member 21 may be a non-woven fabric including heat-fusible fibers that can be bonded through thermal fusion. Meanwhile, in relation to such a portable water purification pouch, Application No. 10-2016-0003770 by the present applicant may be incorporated by reference herein.

Further, in the filter medium for water treatment, it should be noted that the second member 21 and/or the sixth member 23 forming a layer together in addition to the first member may be further provided with an antiviral coating layer.

Meanwhile, the following Table 1 shows amino acid sequences for the above-described antiviral motif and adhesive protein.

TABLE 1

| SEQ ID NO | Amino acid sequence |
|---|---|
| 1 | Lys Lys Lys Tyr Arg Asn Ile Arg Arg Pro Gly |
| 2 | Thr Leu Lys Pro Ile Phe Lys Leu Pro Leu Gly Ile Asn Ile Thr Asn Phe |
| | Arg |
| 3 | Phe His Arg Lys Lys Gly Arg Gly Lys His Lys |
| 4 | Ser Leu Ile Gly Arg Leu |
| 5 | Trp Leu Val Phe Phe Val Ile Phe Tyr Phe Phe Arg Arg Arg Lys Lys |
| 6 | Arg Arg Lys Lys Trp Leu Val Phe Phe Val Ile Phe Tyr Phe Phe Arg |
| 7 | Arg Arg Lys Lys Ala Ala Val Ala Leu Leu Pro Ala Val Leu Leu Ala Leu |
| | Leu Ala Pro |
| 8 | Lys Leu Leu Leu Lys Leu Leu Lys Lys Leu Leu Lys Leu Leu Lys Lys |
| | Lys |
| 9 | Ala Lys Pro Ser Tyr Pro Pro Thr Tyr Lys |
| 10 | Ala Lys Pro Ser Tyr Pro Pro Thr Tyr Lys Ala Lys Pro Ser Tyr Pro Pro Thr |
| | Tyr Lys |
| 11 | Ala Lys Pro Ser Tyr Pro Pro Thr Tyr Lys Ala Lys Pro Ser Tyr Pro Pro Thr |
| | Tyr Lys Ala Lys Pro Ser Tyr Pro Pro Thr Tyr Lys Ala Lys Pro Ser Tyr |
| | Pro Pro Thr Tyr Lys Ala Lys Pro Ser Tyr Pro Pro Thr Tyr Lys Ala Lys |
| | Pro Ser Tyr Pro Pro Thr Tyr Lys |
| 12 | Glu Val His Ala Cys Lys Pro Asn Pro Cys Lys Asn Asn Gly Arg Cys Tyr |
| | Pro Asp Gly Lys Thr Gly Tyr Lys Cys Lys Cys Val Gly Gly Tyr Ser Gly |
| | Pro Thr Cys Ala Cys |
| 13 | Ala Asp Tyr Tyr Gly Pro Lys Tyr Gly Pro Pro Arg Arg Tyr Gly Gly Gly |
| | Asn Tyr Asn Arg Tyr Gly Gly Ser Arg Arg Tyr Gly Gly Tyr Lys Gly Trp |
| | Asn Asn Gly Trp Lys Arg Gly Arg Trp Gly Arg Lys Tyr Tyr Glu Phe Glu |
| | Phe |
| 14 | Ala Asp Tyr Tyr Gly Pro Lys Tyr Gly Pro Pro Arg Arg Tyr Gly Gly Gly |
| | Asn Tyr Asn Arg Tyr Gly Arg Arg Tyr Gly Gly Tyr Lys Gly Trp Asn Asn |
| | Gly Trp Lys Arg Gly Arg Trp Gly Arg Lys Tyr Tyr |
| 15 | Gly His Val His Arg His Arg Val Leu His Lys His Val His Asn His Arg |
| | Val Leu His Lys His Leu His Lys His Gln Val Leu His Gly His Val His |
| | Arg His Gln Val Leu His Lys His Val His Asn His Arg Val Leu His Lys |
| | His Leu His Lys His Gln Val Leu His |
| 16 | Ser Ser Glu Glu Tyr Lys Gly Gly Tyr Tyr Pro Gly Asn Ala Tyr His Tyr |
| | His Ser Gly Gly Ser Tyr His Gly Ser Gly Tyr His Gly Gly Tyr Lys Gly |
| | Lys Tyr Tyr Gly Lys Ala Lys Lys Tyr Tyr Tyr Lys Tyr Lys Asn Ser Gly |
| | Lys Tyr Lys Tyr Leu Lys Lys Ala Arg Lys Tyr His Arg Lys Gly Tyr Lys |
| | Lys Tyr Tyr Gly Gly Ser Ser |
| 17 | Ser Ser Glu Glu Tyr Lys Gly Gly Tyr Tyr Pro Gly Asn Thr Tyr His Tyr |
| | His Ser Gly Gly Ser Tyr His Gly Ser Gly Tyr His Gly Gly Tyr Lys Gly |
| | Lys Tyr Tyr Gly Lys Ala Lys Lys Tyr Tyr Tyr Lys Tyr Lys Asn Ser Gly |
| | Lys Tyr Lys Tyr Leu Lys Lys Ala Arg Lys Tyr His Arg Lys Gly Tyr Lys |
| | Lys Tyr Tyr Gly Gly Gly Ser Ser |
| 18 | Tyr Asp Asp Tyr Ser Asp Gly Tyr Tyr Pro Gly Ser Ala Tyr Asn Tyr Pro |
| | Ser Gly Ser His Trp His Gly His Gly Tyr Lys Gly Lys Tyr Tyr Gly Lys |
| | Gly Lys Lys Tyr Tyr Tyr Lys Phe Lys Arg Thr Gly Lys Tyr Lys Tyr Leu |
| | Lys Lys Ala Arg Lys Tyr His Arg Lys Gly Tyr Lys Lys His Tyr Gly Gly |
| | Ser Ser Ser |
| 19 | Ser Ser Glu Glu Tyr Lys Gly Gly Tyr Tyr Pro Gly Asn Thr Tyr His Tyr |
| | His Ser Gly Gly Ser Tyr His Gly Ser Gly Tyr His Gly Gly Tyr Lys Gly |
| | Lys Tyr Tyr Gly Lys Ala Lys Lys Tyr Tyr Tyr Lys Tyr Lys Asn Ser Gly |
| | Lys Tyr Lys Tyr Leu Lys Lys Ala Arg Lys Tyr His Arg Lys Gly Tyr Lys |
| | Lys Tyr Tyr Gly Gly Gly Ser Ser |
| 20 | Gly Gly Gly Asn Tyr Arg Gly Tyr Cys Ser Asn Lys Gly Cys Arg Ser Gly |
| | Tyr Ile Phe Tyr Asp Asn Arg Gly Phe Cys Lys Tyr Gly Ser Ser Ser Tyr |
| | Lys Tyr Asp Cys Gly Asn Tyr Ala Gly Cys Cys Leu Pro Arg Asn Pro Tyr |
| | Gly Arg Val Lys Tyr Tyr Cys Thr Lys Lys Tyr Ser Cys Pro Asp Asp Phe |
| | Tyr Tyr Tyr Asn Asn Lys Gly Tyr Tyr Tyr Tyr Asn Asp Lys Asp Tyr Phe |
| | Asn Cys Gly Ser Tyr Asn Gly Cys Cys Leu Arg Ser Gly Tyr |
| 21 | Ala Lys Pro Ser Tyr Pro Pro Thr Tyr Lys Ala Lys Pro Ser Tyr Pro Pro Thr |
| | Tyr Lys Ala Lys Pro Ser Tyr Pro Pro Thr Tyr Lys Ala Lys Pro Ser Tyr |
| | Pro Pro Thr Tyr Lys Ala Lys Pro Ser Tyr Pro Pro Thr Tyr Lys Ala Lys |
| | Pro Ser Tyr Pro Pro Thr Tyr Lys Ser Ser Glu Glu Tyr Lys Gly Gly Tyr |
| | Tyr Pro Gly Asn Ala Tyr His Tyr His Ser Gly Gly Ser Tyr His Gly Ser |
| | Gly Tyr His Gly Gly Tyr Lys Gly Lys Tyr Tyr Gly Lys Ala Lys Lys Tyr |
| | Tyr Tyr Lys Tyr Lys Asn Ser Gly Lys Tyr Lys Tyr Leu Lys Lys Ala Arg |
| | Lys Tyr His Arg Lys Gly Tyr Lys Tyr Tyr Gly Gly Ser Ser Ala Lys Pro |
| | Ser Tyr Pro Pro Thr Tyr Lys Ala Lys Pro Ser Tyr Pro Pro Thr Tyr Lys Ala |
| | Lys Pro Ser Tyr Pro Pro Thr Tyr Lys Ala Lys Pro Ser Tyr Pro Pro Thr Tyr |
| | Lys Ala Lys Pro Ser Tyr Pro Pro Thr Tyr Lys Ala Lys Pro Ser Tyr Pro Pro |
| | Thr Tyr Lys |
| 22 | Ala Lys Pro Ser Tyr Pro Pro Thr Tyr Lys Ala Lys Pro Ser Tyr Pro Pro Thr |
| | Tyr Lys Ala Lys Pro Ser Tyr Pro Pro Thr Tyr Lys Ala Lys Pro Ser Tyr |
| | Pro Pro Thr Tyr Lys Ala Lys Pro Ser Tyr Pro Pro Thr Tyr Lys Ala Lys |
| | Pro Ser Tyr Pro Pro Thr Tyr Lys Ser Ser Glu Glu Tyr Lys Gly Gly Tyr |
| | Tyr Pro Gly Asn Thr Tyr His Tyr His Ser Gly Gly Ser Tyr His Gly Ser |
| | Gly Tyr His Gly Gly Tyr Lys Gly Lys Tyr Tyr Gly Lys Ala Lys Lys Tyr |
| | Tyr Tyr Lys Tyr Lys Asn Ser Gly Lys Tyr Lys Tyr Leu Lys Lys Ala Arg |
| | Lys Tyr His Arg Lys Gly Tyr Lys Lys Tyr Tyr Gly Gly Gly Ser Ser Ala |
| | Lys Pro Ser Tyr Pro Pro Thr Tyr Lys Ala Lys Pro Ser Tyr Pro Pro Thr Tyr |
| | Lys Ala Lys Pro Ser Tyr Pro Pro Thr Tyr Lys Ala Lys Pro Ser Tyr Pro Pro |
| | Thr Tyr Lys Ala Lys Pro Ser Tyr Pro Pro Thr Tyr Lys Ala Lys Pro Ser |
| | Tyr Pro Pro Thr Tyr Lys |

The present invention will be described in more detail through the following Examples, but the following Examples are not intended to limit the scope of the present invention, and should be interpreted to help the understanding of the present invention.

### Example 1

After a spinning solution including a fiber-forming component, which is PVDF, was electrospun onto one surface of a second member which is a nonwoven fabric (NamYang Nonwoven Fabric Co., Ltd., CCP30) formed of a core-sheath type composite fiber in which polyethylene with a thickness of about 20 μm and a melting point of about 120° C. serves as a sheath and polypropylene serves as a core, a target to be coated, which is a filter medium in which a first member and a second member, which are PVDF nanofiber webs, were fused was prepared by applying heat and a pressure of 1 kgf/cm² at a temperature of 140° C. by performing a calendering process.

After a first solution containing an antiviral fusion protein prepared through the following preparation example and water and a second solution in which a carbodiimide-based coupling agent and a hydroxy succinimide-based reactive agent were dissolved in ethanol were introduced into an electrospraying device through separate conduits without being mixed, the device was designed such that the first solution and the second solution entered a spraying pack in the electrospraying device while being mixed through a Y-shaped conduit immediately before the spraying pack, and in this case, the first solution and the second solution were allowed to pass through the Y-shaped conduit, such that the concentration of the antiviral fusion protein in the mixed first solution and second solution was 0.1 mg/ml, and the weight ratio of the total weight of the carbodiimide-based coupling agent and the hydroxy succinimide-based reactive agent and the antiviral fusion protein was 1:1. Thereafter, electrospraying was performed on the surface of the filter medium to be coated at a discharge rate of 20 ml/min, a distance of 40 cm between the tip and the collector, at a temperature of 30° C., a relative humidity of 45 RH %, and a voltage of 50 kV applied to the tip.

Thereafter, the target was allowed to pass an IR lamp for initial drying, and then dried with hot air at 70° C. to implement an antiviral filter medium provided with an antiviral coating layer by forming particles of the antiviral fusion protein immobilized on the surface.

### Preparation Example—Preparation of Antiviral Coating Composition

As the antiviral fusion protein, an antiviral fusion protein was prepared in which the carboxyl group terminus of a mussel-derived adhesive protein which is SEQ ID NO: 21 and the amino terminus of the antiviral motif which is SEQ ID NO: 8 were bound. In this case, the antiviral fusion protein was prepared by a recombinant protein production method using Escherichia coli.

Specifically, the first solution was prepared by dissolving the antiviral fusion protein in water. Further, the second solution was prepared by including a carbodiimide-based coupling agent which is 1-[3-(dimethylamino)propyl]-3-ethylcarboimide hydrochloride (EDC), a reactive agent which is N-hydroxysulfosuccinimide (Sulfo-NHS), sodium acetate which is an active component, and water as a solvent, specifically, including EDC and Sulfo-NHS so as to have a weight ratio of 1:1, including sodium acetate such that the weight ratio of sodium acetate based on total weight of EDC and Sulfo-NHS was 1:0.3, and then stirring the resulting mixture. Thereafter, the first solution and second solution prepared were stored at 20° C. and 5° C., respectively.

### Example 2

The antiviral fusion protein was prepared in the same manner as in Example 1, and a filter medium, in which the antiviral fusion protein was immobilized, was prepared by introducing the following antiviral coating composition in which a tyrosine residue of an adhesive protein in the antiviral fusion protein was modified into a DOPA residue into an electrospraying device.

In this case, the modification into a DOPA residue was performed by dissolving the antiviral fusion protein in a buffer solution including 50 mM ascorbic acid to a concentration of 1 mg/ml, and in this case, as the buffer solution, a buffer solution of 40 mM sodium acetate and 20 mM sodium borate was used. Thereafter, the prepared solution was saturated with oxygen while injecting oxygen into the solution for 20 minutes, and then mushroom-derived tyrosinase was added to a final concentration of 35 μg/ml. Thereafter, after mixing and stirring under oxygen conditions for 1 hour, acetic acid was added to a final concentration of 5% to terminate the reaction after the modification reaction into a DOPA residue. Thereafter, the completed reaction solution was desalted and concentrated with a 5% acetic acid solution, and then subjected to a freeze-drying process to obtain an antiviral fusion protein containing a DOPA residue in a powder form. Thereafter, an antiviral coating composition was prepared by dissolving the obtained antiviral fusion protein to a concentration of 0.1 mg/ml using a 40 mM Tris buffer with a pH of 8.2.

### Example 3

A filter medium was prepared in the same manner as in Example 1, and a filter medium, in which the antiviral fusion protein was provided, was prepared using a solution of the antiviral fusion protein dissolved in water as an antiviral coating composition.

### Comparative Example 1

A filter medium was prepared in the same manner as in Example 1, and a filter medium, in which an antiviral motif was provided, was prepared by changing the antiviral fusion protein into the antiviral motif alone.

### Experimental Example 1

The following physical properties were examined for the filter media having surfaces provided with the antiviral coating layer prepared in Examples 1 to 3 and Comparative Example 1, and the results are shown in the following Table 2.

1. Antiviral Performance

A filter medium on which an antiviral coating layer was formed was prepared so as to have a width of 4 cm and a length of 4 cm. Thereafter, after each sample was treated with 200 ml of PED virus (coronaviridae, enveloped RNA virus) and then allowed to stand at 23° C. for 14 hours, the virus located on the sample was collected by adding 800 μl of an inoculation medium which is 1×DMEM (0.3% tryptose phosphate broth, 0.02% yeast extract, 1% antibiotic-antimycotic, 5 μg/ml trypsin) to the sample.

After an inoculation medium containing the collected virus was diluted by decimal dilution, 100 μl of the inoculation medium was inoculated into 5 wells per dilution factor, then adsorbed for 1 hour in a CO₂ incubator, then the inoculum was removed, and 200 μl of a virus culture medium per well was aliquoted to a 96-well plate containing VERO cells at 200×10⁴ cells/well and incubated in an incubator for 5 days. Thereafter, the CPE of the cells was confirmed and the TCID value was calculated, and the resulting values are shown in the following Table 2.

The TCID log conversion value of the viral titer was 5.0000, a positive control was a result evaluated by allowing only 200 μl of the viral stock solution to stand at a temperature of 23° C. for 14 hours, and the TCID log conversion value was 5.0000.

2. Adhesion Performance

For Examples 1 to 3 which had antiviral performance among the filter media with an antiviral surface, impregnating each sample in water at 23° C. and then taking out the sample after 1 minute was defined as one set, and 20 sets were performed, and then the above antiviral performance was evaluated.

3. Storage Stability

After an accelerated aging test according to guidelines for setting the shelf life of medical devices and evaluating stability was performed by the following method, the storage stability of the filter medium with an antiviral surface was evaluated by evaluating the above-described antiviral performance.

Specifically, in order to reproduce the real-time aging of the antiviral filter medium within a shortened period of time, the porous substrate was prepared such that the aging period of each antiviral filter medium was 3 year by storing the porous substrate at an elevated temperature (60° C.) for 3 months.

TABLE 2

| | | | | Comparative |
|---|---|---|---|---|
| | Example 1 | Example 2 | Example 3 | Example 1 |
| Immobilization | Antiviral | Antiviral | Antiviral | Antiviral |
| target | fusion | fusion | fusion | motif |
| | protein | protein | protein | |
| Additional | EDC + | Antiviral | None | None |
| immobilization | Sulfo-NHS | fusion | | |
| method | | protein DOPA | | |
| | | modification | | |
| Antiviral | 3.5 | 3.8 | 4.6 | 5.0 |
| performance | | | | |
| (TCID log | | | | |
| conversion | | | | |
| value) | | | | |
| Adhesion | 3.5 | 3.9 | 5.0 | Not |
| performance | | | | measured |
| (TCID log | | | | |
| conversion | | | | |
| value) | | | | |
| Storage | 3.7 | 4.7 | 5.0 | Not |
| stability | | | | measured |
| (TCID log | | | | |
| conversion | | | | |
| value) | | | | |

As can be confirmed from Table 2, it can be seen that the antiviral performance of the surfaces having the antiviral coating layers formed according to Examples 1 and 2 is effectively present at 90% or more.

In addition, as a result of evaluating the adhesion performance, there was little change in the antiviral performance of Examples 1 and 2, and in particular, there was no change in the antiviral performance according to Example 1, and through this, it can be seen that the method of immobilizing an antiviral fusion protein through Example 1 exhibits excellent adhesion characteristics on the surface.

Furthermore, as a result of the storage stability evaluation, Example 1 showed significantly less reduction in antiviral performance than the other Examples, and through this, it can be seen that the storage stability and the sustainability of the antiviral effect are excellent.

Although one embodiment of the present invention has been described above, the spirit of the present invention is not limited to the embodiments presented in present specification, and a person skilled in the art who understands the spirit of the present invention can easily propose other embodiments by substitutions, changes, deletions, additions, and the like of the constituent elements, but it can be said that those embodiments also fall within the scope of the spirit of the present invention.

1. An antiviral filter medium comprising a first member including fibers provided with an antiviral coating layer formed on part or all of the outer surface of the fibers, wherein the antiviral coating layer includes an antiviral fusion protein in which antiviral motif is bound to an adhesive protein.

2. The antiviral filter medium of claim 1, wherein the antiviral motif targets a protein that binds to a host cell receptor to disable or disrupt the protein or perform a function of disrupting the viral membrane.

3. The antiviral filter medium of claim 1, wherein the adhesive protein is a mussel-derived adhesive protein.

4. The antiviral filter medium of claim 1, wherein the antiviral motif is any one peptide selected from the group consisting of amino acid sequences of SEQ ID NOS: 1 to 8 or a peptide in which one or more amino acid sequences selected from the above group are linked, and
the adhesive protein is any one protein selected from the group consisting of amino acid sequences of SEQ ID NOS: 9 to 22 or a protein in which one or more amino acid sequences selected from the above group are linked.

5. The antiviral filter medium of claim 1, wherein the antiviral coating layer is formed on fibers by aggregating particles formed of an antiviral fusion protein.

6. The antiviral filter medium of claim 5, wherein the antiviral coating layer is formed through an antiviral coating composition comprising an antiviral fusion protein and an aggregation-inducing component comprising a carbodiimide-based coupling agent and a hydroxy succinimide-based reactive agent.

7. The antiviral filter medium of claim 1, wherein the adhesive protein contains a DOPA residue, and the antiviral fusion protein is immobilized on a fiber through the DOPA residue.

8. The antiviral filter medium of claim 7, wherein the DOPA residue is a DOPA residue into which some or all tyrosine residues of the adhesive protein are modified through an enzyme.

9. The antiviral filter medium of claim 1, wherein fibers forming the first member have an average diameter of 0.05 to 1 μm, a basis weight of 2.5 g/m² or less, and an average pore diameter of 2.5 μm or less.

10. The antiviral filter medium of claim 1, further comprising a porous second member disposed on one side or both sides of the first member.

11. The antiviral filter medium of claim 1, further comprising a porous second member disposed on one side of the first member and performing a supporting function and
a porous third member which is disposed on the other side of the first member facing the one side and electrostatically treated.

12. The antiviral filter medium of claim 10, further comprising a porous fourth member disposed between the first member and a third member and comprising a silver wire so as to have an antibacterial function, or comprising a heat-fusible fiber for attaching the first member and the third member.

13. An air filter unit comprising: the antiviral filter medium according to claim 1, which is bent so as to alternately form peaks and valleys in one direction, and
a filter frame surrounding the antiviral filter medium.

14. An air conditioner comprising the antiviral filter medium according to claim 1.
